(19) United States
(12) Patent Application Publication
(10) Pub. No.: US 2007/0184003 A1
(43) Pub. Date: Aug. 9, 2007

(54) Apparatus and method for optimizing hair growth

(76) Inventor: William F. Gaunitz V, Scottsdale, AZ (US)

Correspondence Address: WRIGHT LAW GROUP, PLLC, 7201 WEST OAKLAND, SUITE 2, CHANDLER, AZ, 85226, US

(21) Appl. No.: 11/351,535

(22) Filed: Feb. 9, 2006

(51) Int. Cl. A61K 8/49; A61N 5/06

(52) U.S. Cl. 424/70.11; 607/86

(57) ABSTRACT

A system for enhancing and promoting hair growth addresses the problem of premature or undesirable hair loss with the use of i) laser energy applied to the scalp in conjunction with ii) certain topical applications that can be applied to the scalp on a periodic basis and iii) certain supplements that are ingested orally. The specific applications and combination of ingredients will be determined, at least in part, by the gender of the patient. The apparatus of the present invention directs laser energy, in an optimal range of wavelengths, directly to the scalp in both residential and clinical environments. The topical application described herein, including the gender-based component, preferably combines some combination of Finasteride with one or more of a DHT inhibitor, primrose oil, a hormone balancing multi-vitamin, and a hair growth vitamin, all administered in accordance with a specific protocol. Finally, the supplements provide for detoxification and nutritional enhancement for an individual receiving hair growth treatment.

### BACKGROUND OF THE INVENTION

1. Field of the Invention

The present invention relates generally to the field of medical treatment and more specifically relates to techniques and methods for enhancing and promoting hair growth.

2. Background Art

The loss of hair has traditionally been a problem for a substantial percentage of the population. There are many possible causes of hair loss, include hormones balance, hereditary genes, etc. Pattern hair loss is believed to result from the conversion of testosterone to dihydrotestosterone (DHT) by the 5-alpha-reductase (5AR) enzyme. It is believed that DHT attaches to the hair follicles, causing a negative change in the growth pattern of hair. The hair shrinks in diameter and the hair growth cycle becomes shorter. The result is smaller, thinner, virtually invisible hairs and a shrinking of hair coverage on the scalp. This process is called pattern hair loss. Whether the problem is alopecia (male pattern baldness) or thinning hair, the affected individuals generally find unanticipated hair loss to be distressing and detrimental to their appearance. In addition, the loss of hair will often make individuals appear to be older than they are. For this reason, a variety of attempts have been made to improve an individual's appearance by restoring the appearance of a full head hair.

Early attempts to address this problem focused on the use of hairpieces (i.e. toupees or wigs). While these devices provided some degree of success, they have several drawbacks. In particular, they often have an unnatural appearance that allows them to be easily detected, even from a distance, by other individuals. In order to make one of these devices appear to be the natural hair of the wearer, they are typically constructed in a fairly expensive manner using real human hair that is matched in color to the individual's remaining hair. Unfortunately, many individuals cannot afford a high quality hairpiece such as this, and settle for a lower quality hair piece fabricated from synthetic fibers.

In addition, artificial hairpieces are quite inconvenient to use in certain situations, such as swimming, where they may become damaged or loosened. In this situation, the individual may be embarrassed due to the failure of the device to perform as desired. Most individuals that have experienced undesirable hair loss wish to improve the appearance of an individual's hair without having to resort to hairpieces fastened to an individual's head which are expensive and occasionally prone to failure due to environmental circumstances.

Another attempt to address the problem of undesired hair loss has been to surgically replace missing hair with “hair plugs.” The surgical solution to hair loss overcomes the problems created by the use of hairpieces in that the replacement hair plugs use the real hair of the individual and, if done properly, results in a perfect color match and a natural appearance. In addition, the individual has no particular use restrictions, with regard to swimming and other activities, as may be common with hairpieces. Unfortunately, this method of treating hair loss is relatively expensive and requires the use of medical professionals for the surgical hair transplant procedure. As a result, this procedure may not be feasible for a substantial portion of the public due to its high cost. It would be desirable to have a method of facilitating the growth of hair that was economically available to all or most of those individuals who face premature hair loss.

In addition to hairpieces and surgical transplants, pharmaceutical products have also been developed to encourage or enhance hair growth. These products can take the form of ingestible medications and/or topical skin treatments. The ingestible medications have been proven to encourage hair growth, but they have several significant drawbacks. In particular, they are generally prescription medications that require the cost and inconvenience of visiting a physician. In addition, the fact that they are prescription medications typically means that they will have a higher cost than non-prescription drugs. Perhaps more important than the issue of cost is the potential side effects of ingestible drugs. Quite often, the use of ingestible prescription medications may result in undesirable side effects, such as damage to the individual's liver or other internal organs, and may present other serious side effects. Accordingly, it would be desirable to have a method of stimulating hair growth that did not carry the risks of side effects inherent in ingestible prescription pharmaceutical medications.

Another pharmaceutical solution that has been adopted with some success is the use of topical skin treatments, applied to the scalp area to stimulate hair growth. This type of medication is often similar to prescription medications with the same cost disadvantage of ingestible medications. While some are now available as over the-counter preparations, they typically have a reduced strength and are less effective than are their prescription counterparts. While less expensive than most prescription strength topical treatments, these over-the-counter preparations are also generally less effective. Additionally, some studies have shown that discontinuance of the topical treatment results in a regression in hair to the same state as existed prior to beginning the treatment. Accordingly, topical treatments require a continuing investment in medication to maintain the desired results.

Over the last few decades, scientists have also discovered that laser light sources can be used to stimulate hair growth. Several low power laser devices (including “cold lasers”) have been developed to irradiate an individual's scalp in a clinical setting by utilizing laser energy. While the use of lasers holds promise, laser devices are extremely expensive and the effective use of laser energy to treat hair loss is still in a somewhat embryonic stage. This scenario, coupled with the practical realities of controlling and deploying the laser energy in a safe, effective, and efficient manner, has generally limited the use of laser energy to carefully supervised, and relatively expensive, clinical treatments.

While all of these known methods for treating hair loss have had some limited success, many individuals have not been able to experience the desired results from existing solutions. In many cases, given the significant expense of most medical treatments used to treat hair loss in the clinical environment, many of those afflicted have been simply priced out of the treatment market. Accordingly, without providing innovative, affordable, and enhanced treatment options for promoting and enhancing hair growth in individuals, hair growth opportunities will continue to be sub-optimal.

### SUMMARY OF THE INVENTION

A system for enhancing and promoting hair growth addresses the problem of premature or undesirable hair loss with the use of i) periodic application of laser energy to the scalp in conjunction with; ii) periodic application of certain formulations to the scalp or other areas where hair growth is desired and; iii) certain nutritional supplements in various formulations that are ingested orally. The apparatus of the present invention directs laser energy, in an optimal range of wavelengths, directly to the scalp or other area where hair growth is desired in both clinical and non-clinical environments. The topical application described herein, including a gender-based component, preferably combines Finasteride with one or more of a DHT inhibitor, primrose oil, a hormone balancing multi-vitamin, and a hair growth vitamin, all administered in accordance with a specific protocol. Finally, the supplements provide nutritional enhancement for an individual receiving hair growth treatment.

### BRIEF DESCRIPTION OF THE DRAWINGS

The preferred embodiments of the present invention will hereinafter be described in conjunction with the appended wherein like designations denote like elements and:

FIG. 1 is a block diagram representing the three components of a system for optimizing hair growth in accordance with a preferred exemplary embodiment of the present invention;

FIG. 2 is a schematic representation of the laser energy component of a system for optimizing hair growth in accordance with a preferred exemplary embodiment of the present invention;

FIG. 3 is a schematic representation of a nutritional supplementation component of a system for optimizing hair growth in accordance with a preferred exemplary embodiment of the present invention;

FIG. 3A is a schematic representation of the detoxifying supplement component of a system for optimizing hair growth in accordance with a preferred exemplary embodiment of the present invention;

FIG. 4 is a schematic representation of the topical treatment portion of a system for optimizing hair growth in accordance with a preferred exemplary embodiment of the present invention; and

FIG. 5 is a flow chart for a method of implementing a system for optimizing hair growth in accordance with a preferred exemplary embodiment of the present invention.

### DETAILED DESCRIPTION OF THE INVENTION

A system for enhancing and promoting hair growth addresses the problem of premature or undesirable hair loss with the use of i) laser energy applied to the scalp in conjunction with ii) certain topical applications that can be applied to the scalp on a periodic basis and iii) certain supplements that are ingested orally. The specific applications and combination of ingredients will be determined, at least in part, by the gender of the patient. The apparatus of the present invention directs laser energy, in an optimal range of wavelengths, directly to the scalp in both residential and clinical environments. The topical application described herein, including the gender-based component, preferably combines some combination of Finasteride with one or more of a DHT inhibitor, primrose oil, a hormone balancing multi-vitamin, and a hair growth vitamin, all administered in accordance with a specific protocol. Finally, the supplements provide for detoxification and nutritional enhancement for an individual receiving hair growth treatment.

The most preferred embodiments of the present invention include various therapies that, when combined together, provide an optimal opportunity for hair regeneration and hair growth. The methodology is specifically adapted to optimize results for both men and women and, in the most preferred embodiments of the present invention, is specifically adapted to patient gender. The most preferred embodiments of the present invention include three separate “components” or treatment elements that are combined to achieve the desired results. Those skilled in the art will recognize that the various components may be used independently, simultaneously, separately and/or together in various combinations, depending on a specific patient's needs and goals for hair growth.

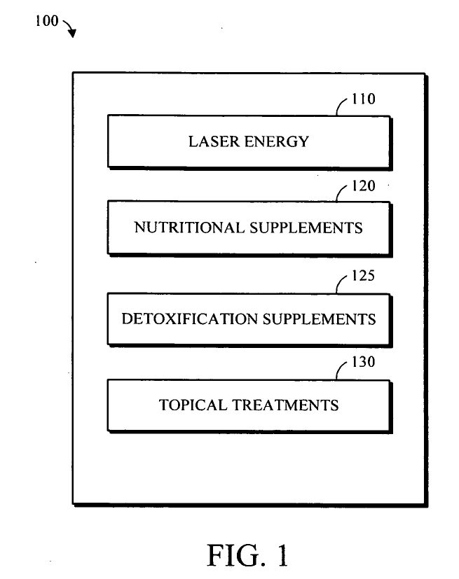

Referring now to FIG. 1, a block diagram for a system 100 comprising four separate components for enhanced hair growth in accordance with a preferred embodiment of the present invention is depicted. As shown in FIG. 1, system 100 includes: a laser energy component 110; a nutritional supplement component 120; a detoxification component 125; and a topical treatment component 130.

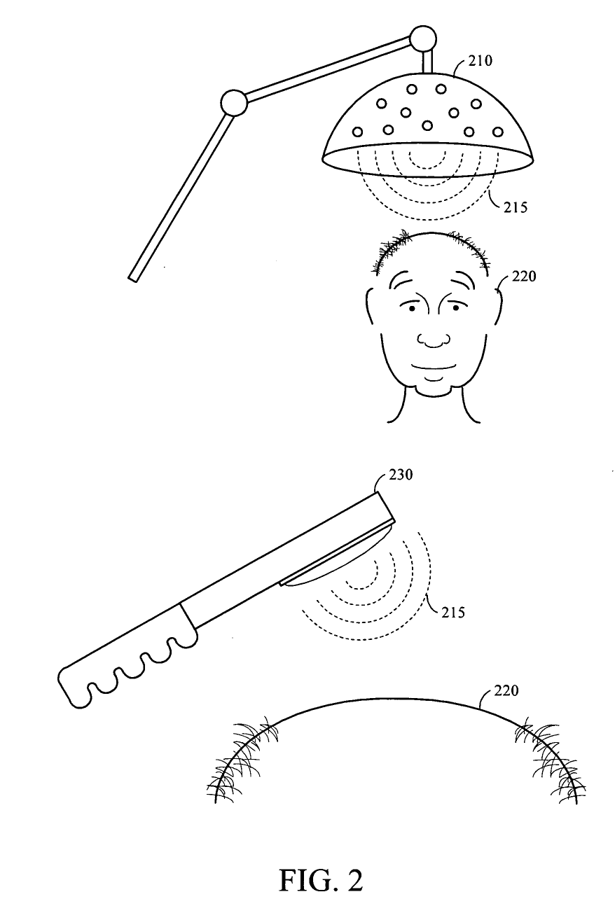

Referring now to FIG. 2, the use of laser energy component 110 of FIG. 1 is described in further detail. Laser energy component 110 is most preferably included in all treatment phases and comprises the application of a relatively low power laser light to the scalp. As previously mentioned, laser energy component 110 is most preferably combined with topical scalp treatments and nutritional supplements. In the most preferred embodiments of the present invention, laser light, generated by a class 3A laser device, with a frequency range of approximately 600 nm-690 nm is applied to the scalp. The most desired range for the laser light being applied to the scalp is in the range of 620 nm-645 nm. While not preferred, other frequencies, outside of the most preferred range, may also be employed with varying results. In general, the higher the power of the laser, the less exposure time that will be required for a standard treatment. Additionally, the pulse rate may also be considered as a relevant factor in determining the exact amount of laser treatment to be applied to the scalp. Suitable representative lasers are offered by Erchonia Medical, Inc., Crown Laser of Australia, and Transform Technologies AB.

In the clinical environment, laser energy component 110 is provided by use of a low power laser unit 210. Laser unit 210 provides laser energy 215 to the scalp of patient 220. The use of laser unit 210 to provide laser energy 215 in a clinical setting is well known to those skilled in the art.

During the application of laser energy 215 to the scalp, care should be exercised so as to prevent overexposure of the scalp to laser energy 215. The periodic application of laser energy 215 in a clinical setting will continue for a period of time that may range from a few months to a few years, depending on the needs of the patient and the desired results. The present invention contemplates and comprises, in the most preferred embodiments, application of laser energy 215 to the scalp in both a clinical environment as well as a residential environment. Patient 220 will visit the treatment facility on a weekly or biweekly basis with the scalp being exposed to laser light for a period of 5 minutes to 45 minutes.

In the most preferred embodiments of the present invention, patient 220 will also be provided with a small handheld laser unit 230 that will used to provide laser energy 215 in a residential or non-clinical environment. The use of laser unit 230 provides patient 220 with an opportunity to conduct additional treatment sessions in the residential environment. In the most preferred embodiments of the present invention, patient 220 will use the handheld laser unit to apply laser energy to the scalp for a period of approximately 1 minute to 10 minutes on a daily basis. In the most preferred embodiments of the present invention, the handheld laser unit utilized by patient 220 will emit laser, light with a wavelength of 635 nm and with a frequency pulse rate of 8.967 Khertz. With the use of both clinical laser systems and residential hand-held units, a wide variety of treatment options and protocols are available. Accordingly, the exact laser treatment selected is not as critical as incorporating a laser treatment component in conjunction with the other components in the present invention to achieve the most successful results.

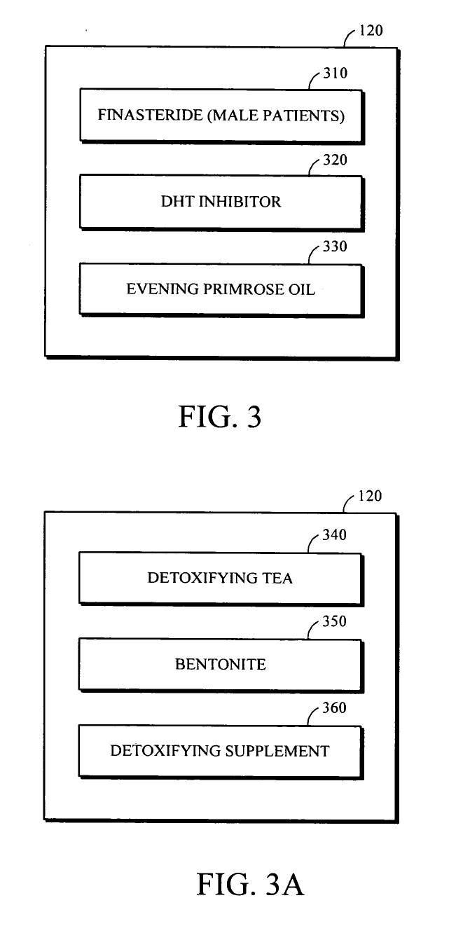

Referring now to FIG. 3, nutritional supplement component 120 of FIG. 1 is described in further detail. While laser energy component 110 is considered the primary treatment component for enhancing hair growth, the inclusion of nutritional supplement component 120 can be added to the treatment regime to further enhance the probability of achieving the desired hair growth results. Nutritional supplement component 120 comprises Finasteride 310; DHT Inhibitor 320; Evening Primrose Oil 330; Detoxifying Tea 340; and Bentonite 350. The ingestion of the various supplements contained in nutritional supplement component 120 may stimulate and enhance hair growth. In general, the nutritional supplements contained in nutritional supplement component 120 are administered after considering the gender of the patient. Additional gender-based treatment elements may also be included in nutritional supplement component 120.

Those skilled in the art will recognize that while the various elements of nutritional supplement component 120 are most preferably used in concert, each individual supplement may be used without regard to the other supplements. However, the most preferred embodiments of the present invention will use all of the nutritional supplements shown in FIG. 3 so as to achieve the maximum benefit possible. Additionally, other nutritional supplements may be incorporated into nutritional component 120 without departing from the spirit and scope of the present invention and no such supplement is excluded by it omission in FIG. 3.

During the primary treatment phase incorporating laser energy component 110, male patients will also ingest Finasteride in an amount of 0.25 mg-10 mg daily. The most preferred embodiments of the present invention provide for the ingestion of Finasteride in an amount of 0.5 mg-5 mg daily. Once again, those skilled in the art will recognize that the prescribed ranges are not limiting, but illustrative for purposes of disclosing the best mode of the invention. Additionally, male patients will ingest one or more vitamins.

Since DHT can be a cause of undesired hair loss, a DHT inhibitor 320 is incorporated into nutritional supplement component 120. DHT inhibitor 320 is most preferably provided in capsule form and comprises a blend of supplements that are most preferably taken twice a day, in the morning and in the evening. Each capsule of DHT inhibitor 320 suitably comprises: Saw Palmetto Berry Extract (standardized from 45%-95%) (400 mg); Green Tea Extract (200 mg); Alpha Lipoic Acid (200 mg); Stinging Nettle Root (240 mg); L-Lysine (100 mg); Grape Seed Extract (100 mg); Pygeum Bark Extract (75 mg); Pumpkin Seed Extract 4:1 (50 mg); Lycopene (25 mg); and Zinc (25 mg).

For female patients, nutritional supplementation will most preferably include evening primrose oil (EPO), a DHT inhibitor, and a hormone balancing multi-vitamin. Additionally, a multi-vitamin specifically formulated for women, including the component nutrients as shown below, may also be used.

| | Amount | | % Daily |
|---|---|---|---|
| Vitamin | Per Serving | | Value |
| Vitamin A (100% as Natural Beta- | 10,000 | IU | 200% |
| Carotene ™) | | | |
| Vitamin B1 (from Thiamine HCI) | 25 | mg | 1670% |
| Vitamin B2 (Riboflavin) | 25 | mg | 1470% |
| Vitamin B3 (Niacin) | 50 | mg | 250% |
| Vitamin B5 (Panthenic Acid from D- | 50 | mg | 500% |
| Calcium Pantothenate) | | | |
| Vitamin B6 (from Pyridoxine HCL) | 50 | mg | 2500% |
| Vitamin B7 (Biotin) | 300 | mcg | 100% |
| Vitamin B8 (Inositol) | 25 | mg | † |
| Vitamin B9 (Folic acid/Folate) | 800 | mcg | 200% |
| Vitamin B-12 (as Cyanocobalamin) | 200 | mcg | 3330% |
| Vitamin C (from Calcium Ascorbate) | 300 | mg | 500% |
| Vitamin D (as Ergocalciferol) | 400 | IU | 100% |
| Vitamin E (as d-alpha Tocopheryl Succinate) | 200 | IU | 670% |
| Vitamin K (as Phytonadione) | 80 | mcg | 100% |

Additional nutritional components in the multi-vitamin may also include Calcium, Magnesium, Zinc, Iron, Soy Isoflavones, Spirulina, Multi-Enzyme Blend, and Chaste Tree Berry. Those skilled in the art will recognize that these nutritional components are not necessarily exhaustive or exclusive and other nutritional components may be added as well.

Evening Primrose Oil 330 is most preferably taken orally in the amount of 2500 mg daily for all patients.

Referring now to FIG. 3A, detoxification supplements in accordance with a preferred embodiment of the present invention include Detoxifying Tea 340. Detoxifying Tea 340 is a tea-like drink that is most preferably prepared using the following ingredients in the following proportions: Burdock root (5 parts), Dandelion root (5 parts), licorice root (2.5 parts), cinnamon chips (1 part), and orange peel (1 part). The dry ingredients for Detoxifying Tea 340 are typically provided in powdered form and the appropriate amounts of each ingredient is combined in a pan with approximately 36 ounces of boiling water and then simmered together for approximately 20 minutes over low heat. The liquid is then strained to yield about 36 ounces of tea which is typical of a single day's serving of Detoxifying Tea 340 (three servings of approximately 12 ounces each). Detoxifying Tea 340 may be consumed either hot or cold and is most preferably ingested three times daily during the first four weeks of treatment.

Those skilled in the art will recognize that the detoxifying elements specified in Detoxifying Tea 340 may be provided by other means as well. For example, similar results may be obtained by ingesting an encapsulated detoxifying supplement. One such suitable encapsulated detoxifying supplement is the Cleansemax™ product offered by Advanced Naturals™. By ingesting Detoxifying Tea 340 and/or an encapsulated detoxifying supplement as set forth herein, the process of hair growth can be further optimized.

Additionally, in the most preferred embodiments of the present invention, the patient will also consume Bentonite Clay 350 for the first four weeks to eight weeks of the treatment program. Bentonite is a type of volcanic ash that is released in a fine steam or mist when a volcano erupts. Bentonite contains many minerals, including montmorillonite ((Na, Ca) (Al, Mg)₆(Si₄O₁₀)₃(OH)₆—nH₂O, Hydrated Sodium Calcium Aluminum Magnesium Silicate Hydroxide), and serves to mineralize the soil surrounding the volcano. Bentonite Clay 350 can be mined from veins which are usually two to three feet wide and deep, but may be many yards in length.

The value of the mineral montmorillonite lies in its ability to adsorb many times its own weight and volume when prepared in an aqueous medium. Montmorillonite has a predominantly negative charge that is capable of attracting many kinds of positively charged particles. The negative charge enables the montmorillonite to pick up positively charged, toxic material from the alimentary canal which is then expelled in the feces. The adsorption is a rapid process and can quickly neutralize allergens before they become attached to blood cells, thus preventing an allergic reaction. There is no evidence that bentonite has any chemical action on the body. Its power is purely physical.

Bentonite Clay 350 most preferably comprises a United States Pharmacopeia (USP) grade colloidal bentonite containing the active constituent montmorillonite that is super-refined with demineralized water as a delivery vehicle for consumption. While Bentonite Clay 350 may be the only component of nutritional supplement 120, it is most effective when consumed in conjunction with Detoxifying Tea 340 and/or evening primrose oil 330. Two or three tablespoons of Bentonite Clay 350 is most preferably mixed in an 8 oz glass of water and consumed twice a day; 45 minutes before breakfast on an empty stomach and 15 minutes before dinner on an empty stomach.

In yet another preferred embodiment of the present invention, the detoxification portion of the present invention may be accomplished by having the patient ingest Detoxifying Tea 340 and Bentonite Clay 350 as explained above for a period of one month. After completing the one month regimen of Detoxifying Tea 340 and Bentonite Clay 350, the patient will then begin a one month regiment of ingesting a detoxifying vitamin supplement 360. Following the one month vitamin program, the patient will then enter a “maintenance phase” that contains ingredients similar to the ingredients contained in Detoxifying Tea 340 or the Cleansmax® vitamin product.

Finally, in addition to laser energy component 110, nutritional supplements component 120 and detoxification component 125 described above, the most preferred embodiments of the present invention will also incorporate topical treatments component 130. Topical treatments component 130 comprises a variety of topical treatments applied to the scalp or other area where hair growth is desired with the formulations being specifically selected to enhance the growth of hair.

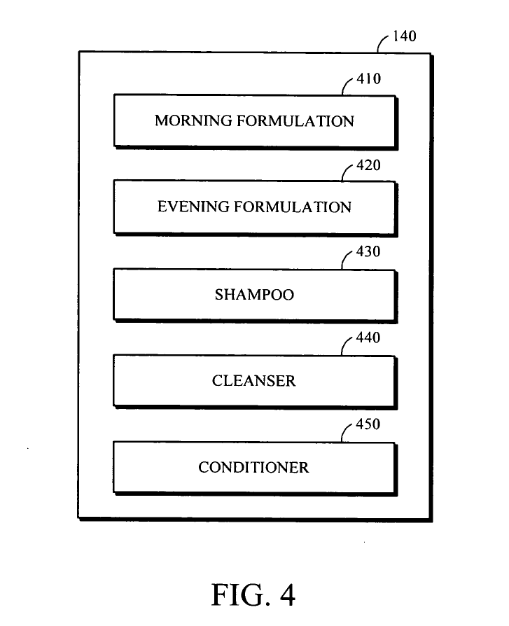

Referring now to FIG. 4, topical treatment component 130 of FIG. 1 is described in further detail. The most preferred embodiments of the present invention comprise a topical application that is most preferably applied to the scalp in the morning (morning formulation 410) when the patient awakens and a separate topical application (evening formulation 420) that is applied in the evening, most preferably applied to the scalp just prior to when the patient retires for the night. Morning formulation 410 most preferably comprises a compound of 5% Azeliac Acid and 5% Minoxidil with beneficial ranges from 1% to 10% of each (1 ml applied daily in the morning). Evening formulation 420 most preferably comprises a compound containing 0.01% retinoic acid and 5% Minoxidil with beneficial ranges from 1% to 10% of each (1 ml applied daily in the evening). While specified as being applied approximately 12 hours apart for the most preferred embodiments of the present invention, Morning formulation 410 and Evening formulation 420 may be applied at approximately the same time or at other intervals as well.

In the most preferred embodiments of the present invention, morning formulation 410 is a combination of Minoxidil™ and Azelaic Acid with a formulation that will suitably comprise:

natural glycerin—20 ml

Minoxidil™ powder—5 grams

Azelaic Acid—(5 grams plus weight necessary to achieve 100% activity)

The glycerin is combined with the Minoxidil™ and the Azelaic Acid to form a paste. The paste is then placed in a glass beaker on a stir plate and denatured alcohol (for topical use only) is added to 80% of the total volume and stirred until the solution is transparent (it will retain a slightly yellow coloration). Then denatured alcohol will be added to reach the target volume of 100 ml.

In the most preferred embodiments of the present invention, evening formulation 420 is a combination of Minoxidil™ and Retinoic Acid with a formulation that will suitably comprise:

natural glycerin—20 ml

Minoxidil™ powder—5 grams

Retinoic Acid—0.01 grams

The glycerin is combined with the Minoxidil™ and the Retinoic Acid to form a paste. The paste is then placed in a glass beaker on a stir plate and denatured alcohol (for topical use only) is added to 80% of the total volume and stirred until the solution is transparent (it will retain a slightly yellow coloration). Then denatured alcohol will be added to reach the target volume of 100 ml.

In addition to the daily topical application of morning formulation 410 and evening formulation 420 described above, a daily shampoo/condition/cleanse process provides for the introduction of beneficial compounds that may be applied to the scalp as well. The steps for the shampoo/cleanse/condition process includes the application of a unique shampoo 430, a unique cleanser 440, and a unique conditioner 450, all of which are applied to the scalp or other area where hair growth is desired.

While various topical treatments are described herein in conjunction with shampoo 430, cleanser 440, and conditioner 450, another important consideration is, to the extent possible, the avoidance of certain ingredients that may inhibit hair growth and/or accelerate hair loss. Some of these undesirable components include, but are not limited to, Polypylene Glycol, Sodium Lauryl Sulfate, Sodium Laureth Sulfate, Diethanolamine (DEA), Moneothanolamine (MEA), Triethanoloamine (TEA), DMDM and Imidazolidinyl (urea), Polyethylene Glycol, and Parabens. The most preferred embodiments of the present invention will typically exclude the use of these and similar compounds.

Shampoo 430 is applied to and massaged into the hair and surrounding scalp area for approximately one minute, is rinsed away. This is followed by another application of Shampoo 430 to the same area again, followed by a formal rinse. Shampoo 430 is used to preliminary cleanse the hair and surrounding scalp area. After the second rinse, the application of Shampoo 430 is followed by the application of Cleanser 440 to the hair and surrounding scalp area. Cleanser 440 is massaged into the hair and surrounding scalp area and allowed to remain in place for approximately one minute and is then rinsed away. Finally, Conditioner 450 is then applied to the hair and surrounding scalp area. Conditioner 450 is gently massaged into the hair and surrounding scalp area for approximately two minutes, then rinsed away.

In the most preferred embodiments of the present invention, shampoo 430 comprises Purified Water, Cocaine Propyl Betaine, Vegetable Glycerin, Organic Peppermint Oil Organic Fennel Extract. Hops Extract Balm Mint Extract. Olive leaf Extract. Organic Ginger Extract, Organic Mistletoe Extract, Allantoin, Citric Acid. Niacin. Grape Seed Extract. Organic Comphrey Extract. Organic lemon Grass Extract. Organic Burdock Root Extract. Organic Sage Extract and Organic Rosemary Extract.

In the most preferred embodiments of the present invention, Cleanser 440 comprises Purified Water, SD Alcohol, Hydrolyzed Wheat Protein, Dimethicone Copolyol, Ploysorbate 80, Oil of Spearmint, Menthol and FD&C Blue #1.

In the most preferred embodiments of the present invention, conditioner 450 comprises: Purified Water, vegetable glycerin, Soybean Oil, Vegetable oil, Organic Oil of Spearmint, Panthenol, Carotene, Vitamin E (D-alpha Tocopherol) Olive Leaf Extract, Organic Ginger Extract, Grape Seed Extract, Organic Chamomile Extract, Organic Comphery Root Extract and Rosemary Extract.

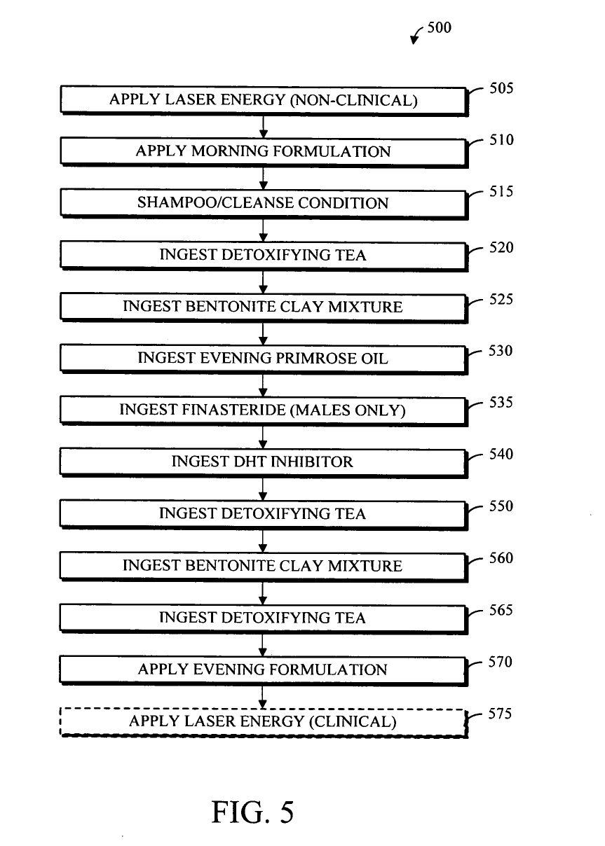

Referring now to FIG. 5, a flow chart for a method 500 of enhancing hair growth in accordance with a preferred embodiment of the present invention in depicted. As shown in FIG. 5, method 500 includes a series of steps that, taken together, provide for incorporating all three components of system 100 from FIG. 1. For the most preferred embodiments of the present invention, and with the exception of the application of laser energy in the clinical environment (step 575), the steps of method 500 are meant to be performed daily, the number of times indicated, and may suitably be performed in the approximate sequence illustrated.

Laser energy is applied to the scalp in a non-clinical environment, typically the home or office of the patient (step 505). The morning formulation of nutritional supplements is applied to the scalp (step 510) by the patient and the hair and surrounding scalp area are treated by the application of the shampoo, cleanser, and conditioner (step 515). Then, the patient will ingest the detoxifying tea (step 520) and the bentonite clay mixture (step 525), preferably prior to breakfast and on an empty stomach. Next, the evening primrose oil is ingested (step 530) and male patients will also ingest Finasteride (step 535) while patients of both genders will ingest the DHT inhibitor (step 540). Once again, the patient will ingest the detoxifying tea (step 550) and the bentonite clay mixture (step 560). Later on, preferably in the evening, the patient will ingest the final dose of the detoxifying tea (step 565) and apply the evening formulation (step 570). Additionally, once every week or every other week, the patient will receive the application of laser energy in a clinical environment (step 575).

While the steps of method 500 are necessarily presented in a certain order, it should be noted that deviations from the specific sequence of steps illustrated in FIG. 5 are contemplated and considered to be in keeping with the spirit and scope of the present invention. For example, the application of laser energy in the non-clinical environment (step 505) may be performed at any time of the day without negatively affecting the overall results of implementing method 500. Similarly, the shampoo/cleanse/condition process (step 515) may be performed in the morning or the evening, without detracting from the value of the shampoo/cleanse/condition process (step 515). The order of the steps set forth in FIG. 5 is simply designed to illustrate the best mode of practicing the invention, as presently understood by the inventors.

In summary, the present invention provides for the broad application of a unique process for evaluating enhancing hair growth in a wide variety of individuals. While the various preferred embodiments of the present invention have been described in conjunction with specific examples, those skilled in the art will appreciate that the apparatus and methods of the present invention are suitable for deployment in a wide variety of circumstances and environments. Lastly, it should be appreciated that the illustrated embodiments are preferred exemplary embodiments only, and are not intended to limit the scope, applicability, or configuration of the present invention in any way. Rather, the foregoing detailed description provides those skilled in the art with a convenient road map for implementing a preferred exemplary embodiment of the present invention.

For example, those skilled in the art will recognize that the expanded use of the laser light, in both a clinical and residential setting, without any nutritional supplements or topical application will provide some measure of improved hair growth. Similarly, the use of nutritional supplements, without the application of laser light, will also provide some measure of success for hair growth. In like fashion, it is anticipated that some limited success could also be achieved by the use of topical treatments alone. However, the most preferred embodiments of the present invention will incorporate the use of laser light therapy, nutritional supplements, and topical scalp treatments to maximize the desired results. Accordingly, it should be understood that various changes may be made in the components, the applications and implementation methodology described in the preferred exemplary embodiments without departing from the spirit and scope of the present invention as set forth in the appended claims.

1. A system to stimulate hair growth comprising:
a laser apparatus, said laser apparatus generating a laser light, said laser light generating a plurality of light waves, said plurality of light waves being directed towards a scalp;
at least one of a first topical treatment and a second topical treatment applied to said scalp, said first topical treatment comprising a combination of Minoxidil™ and Retinoic Acid and said second topical treatment comprising a combination of Minoxidil™ and Azelaic Acid; and
at least one of a detoxification supplement and a nutritional supplement.

2. The system of claim 1 wherein said plurality of light waves have a wavelength in the range of 620-645 nanometers.

3. The system of claim 1 wherein said first topical treatment and said second topical treatment are applied at approximately the same time.

4. The system of claim 1 wherein said first topical treatment and said second topical treatment are applied approximately 12 hours apart.

5. The system of claim 1 wherein said first topical treatment and said second topical treatment further comprises denatured alcohol.

6. The system of claim 1 wherein said first topical treatment and said second topical treatment further comprises glycerin.

7. The system of claim 1 further comprises a third topical treatment, said third topical treatment comprising at least one of:
a topically applied shampoo;
a topically applied cleanser; and
a topically applied conditioner.

8. The system of claim 1 wherein said detoxifying supplement comprises at least one of:
a burdock root component;
a dandelion root component;
a licorice root component;
a cinnamon chip component; and
an orange peel component.

9. The system of claim 1 wherein said detoxifying supplement comprises an ingested bentonite clay supplement.

10. A method of enhancing hair growth comprising the steps of:
applying laser energy to a scalp; and
applying at least one of a first topical treatment and a second topical treatment to said scalp, said first topical treatment comprising a combination of Minoxidil™ and Retinoic Acid and said second topical treatment comprising a combination of Minoxidil™ and Azelaic Acid.

11. The method of claim 10 wherein said step of applying at least one of a first topical treatment and a second topical treatment to said scalp comprises the step of applying both said first topical treatment and said second topical treatment to said scalp.

12. The method of claim 10 further comprising the step of applying at least one of a shampoo, a cleanser and a conditioner to said scalp.

13. The method of claim 10 further comprising the steps of:
applying a shampoo to said scalp;
applying a cleanser to said scalp;
applying a conditioner to said scalp.

14. The method of claim 10 further comprising the step of ingesting a supplement.

15. The method of claim 14 wherein said supplement comprises at least one of:
a bentonite clay component;
an evening primrose oil component;
a burdock root component;
a dandelion root component;
a licorice root component;
a cinnamon chip component; and
an orange peel component.

16. The method of claim 14 wherein said nutritional supplement comprises:
a bentonite clay component;
an evening primrose oil component;
a burdock root component;
a dandelion root component;
a licorice root component;
a cinnamon chip component; and
an orange peel component.

17. The method of claim 14 wherein said nutritional supplement is a gender-based nutritional supplement.

18. The method of claim 10 wherein said step of applying laser energy to a scalp comprises the step of applying laser energy to said scalp in both a clinical and a residential environment.

19. A method of enhancing hair growth comprising the steps of:
applying laser energy to a scalp in a clinical setting;
applying laser energy to a scalp in a residential setting;
applying at least one of a first topical treatment to said scalp, said first topical treatment comprising a combination of Minoxidil™ and Retinoic Acid;
applying a second topical treatment to said scalp, said second topical treatment comprising a combination of Minoxidil™ and Azelaic Acid;
ingesting a nutritional supplement, said nutritional supplement comprising at least one of:
a bentonite clay component;
an evening primrose oil component;
a burdock root component;
a dandelion root component;
a licorice root component;
a cinnamon chip component; and
an orange peel component;
applying a shampoo to said scalp;
applying a cleanser to said scalp; and
applying a conditioner to said scalp.

20. The method of claim 20 wherein said nutritional supplement comprises:
an evening primrose oil component;
a burdock root component;
a dandelion root component;
a licorice root component;
a cinnamon chip component;
an orange peel component; and
bentonite clay component.
